(19) United States
(12) Patent Application Publication
(10) Pub. No.: US 2004/0110774 A1
(43) Pub. Date: Jun. 10, 2004

(54) Methods and formulations of using A1 adenosine receptor antagonists and P2x purinoceptor antagonists for the treatment and prevention of immune system disorders

(76) Inventor: Constance Neely Wilson, Raleigh, NC (US)

Correspondence Address: Shawna Cannon Lemon, Myers Bigel Sibley & Sajovec, P.A., P. O. Box 37428, Raleigh, NC, 27627, US

(21) Appl. No.: 10/713,860

(22) Filed: Nov. 17, 2003

Related U.S. Application Data
(63) Continuation-in-part of application No. PCT/US02/15854, filed on May 17, 2002.
(60) Provisional application No. 60/292,072, filed on May 18, 2001.

(51) Int. Cl. A61K 39/395; A61K 31/52

(52) U.S. Cl. 514/263.1; 424/143.1

(57) ABSTRACT

A₁ adenosine receptor antagonists and P_{2X} purinoceptor antagonists are useful in the treatments of disorders of the immune system, which include HIV infection, AIDS, and adenosine deaminase deficiency-dependent severe immunodeficiency disease (ADA SCID).

### RELATED APPLICATIONS

This application is a continuation-in-part of PCT International Application No. PCT/US02/15854, filed May 17, 2002, entitled Methods and Formulations of Using A₁ Adenosine and P_{2X} Purinoreceptor Antagonists, which claims priority from U.S. Provisional Patent Application Serial No. 60/292,072, filed May 18, 2001, entitled Methods and Formulations of Using A₁ Adenosine Receptor Antagonists and P_{2X} Purinoceptor Antagonists for the Treatment and Prevision of Immune System Disorders, the contents of which are hereby incorporated herein by reference.

### FIELD OF THE INVENTION

The present invention relates to methods for the treatment and prevention of disorders of the immune system, and in particular for the treatment and prevention of HIV infection and AIDS.

### BACKGROUND OF THE INVENTION

Purinergic receptors can be classified into the P₁ (adenosine) receptors and the P₂ (adenosine 5′ triphosphate) receptors. Adenosine receptors can further be delineated into major subclasses, the A₁, A₂ (A₂ₐ and A_{2b}) and A₃ adenosine receptors. These subtypes are differentiated by molecular structure, radioligand binding profiles, pharmacological activity and signal transduction mechanisms. Binding of adenosine (a naturally occurring nucleoside) to specific adenosine receptors leads to either stimulation (A₂-receptor activation) or inhibition (A₁-receptor activation) of adenylate cyclase activity, resulting in an increase or decrease of intracellular cAMP, respectively. Most tissues and cell types possess either the A₁ or A₂ receptor, or both. Specific A₁, A₂, and A₃ adenosine receptor antagonists and agonists are known. See, e.g., Trivedi et al., Structure-Activity Relationships of Adenosine A₁ and A₂ Receptors, in: Adenosine and Adenosine Receptors, M. Williams, Ed., Humana Press, Clifton, N.J., USA (1990); Jacobson et al., J. Medicinal Chem. 35, 407 (1992); Fredholm et al., Pharm. Rev. 46, 143 (1994); Jacobson, Abstracts from Purines '96, Drug Dev. Res., March 1996, page 112.

Based on potency profiles of structural analogues for ATP, ATP-sensitive (P₂) purinoceptors have been subclassified into P_{2X} and P_{2Y} purinoceptors. Seven P_{2X}receptors belong to the most simple transmitter-gated ion channel family, and are found throughout the body in the nervous system (central and peripheral), heart, and on smooth muscle, platelets, lymphocytes and macrophages. Khakh et al., Pharmacol. Rev. 53,107 (2001). P_{2X} receptors are located on vascular smooth muscle cells and mediate vasoconstriction, while P_{2Y} receptors are generally located on endothelial cells and mediate vasodilation. Burnstock and Kennedy, Gen. Pharmacol. 16, 433 (1985); Ralevic et al., Br. J. Pharmacol. 103,1108 (1991).

Inflammatory cells, including monocytes and alveolar macrophages, express the A₁, A₂ and A₃ adenosine receptor subtypes. Eppell et al., J. Immunology 143, 4141 (1989); Lapin and Whaley, Clin. Exp. Immunol. 57,454 (1984); Saijadi, et al., J. Immunol. 156, 3435 (1996). The presence of A₁ adenosine receptors on human monocytes/macrophages is known. See J. E. Salmon, J. Immunology 151, 2775 (1993). Mature monocytes enter the circulatory system from the bone marrow; some monocytes enter tissues and develop into macrophages in the spleen, lymph nodes, liver, lung, thymus, peritoneum, nervous system, skin and other tissues. Both monocytes and macrophages play a role in inflammatory responses, and secrete various proteins active in immune and inflammatory responses, including tumor necrosis factor (TNF) and interleukin-1 (IL-1). Upon stimulation, monocytes and macrophages can generate various oxygen metabolites that are toxic to both pathogens and normal cells, including superoxide anion and H₂O₂. A₁ adenosine receptors are also present on human lymphocytes and PMNs.

A₂ adenosine receptors are present on human B and T (OKT4+ and OKT8+) lymphocytes, PMNs, monocytes, basophils, and platelets, where they inhibit superoxide anion generation by PMNs, histamine release from human basophils, and platelet aggregation. A₂ₐ receptors have been identified as the predominantly expressed subtype of adenosine receptors in T cells. It has been suggested that A₂ₐ receptors are involved in adenosine-mediated immunosuppression under adenosine deaminase (ADA) deficiency conditions in vivo. M. Koshiba et al., J. Biol. Chem. 272, 25881 (1997).

Accumulation of adenosine and of deoxyadenosine in the absence of adenosine deaminase activity (ADA) results in lymphocyte depletion and in severe combined immunodeficiency (ADA SCID). Patients with adenosine deaminase deficiency and severe combined immunodeficiency exhibit markedly impaired lymphocyte proliferation and antibody synthesis. These patients have also been found to have an increased intracellular concentration of ATP and elevated levels of plasma adenosine. It has been determined that immunological defects in severe combined immunodeficiency and adenosine deaminase deficiency may result in part from excessive cyclic AMP synthesis associated with overstimulation of the adenosine receptor-adenyl cyclase pathway. See A. L. Schwartz et al., Clin. Immunol. Immunopathol. 9, 499-505 (1978). It has also been determined that adenosine deaminase can prevent the accumulation of adenosine in thymocytes. In certain studies, thymic adenosine concentrations of mice treated with an ADA inhibitor were elevated over 30-fold, and adenosine concentrations in mice treated with an ADA inhibitor are sufficient to cause adenosine receptor-mediated thymocyte apoptosis in vitro, suggesting that adenosine accumulation could play a role in ADA-deficient severe combined immunodeficiency. R. Resta et al. J. Clin. Invest. 99, 676-683 (1997). In ADA SCID and severe immunodeficiency disease (SCID), however, there is a lack of correlation between ADA replacement treatment and clinical effects.

It has been suggested that both P₂ and P₁ purinoceptors, via transmembrane signaling, play an integral role in differentiation of immature CD4+CD8+thymocytes into CD8+CD4− and CD4+CD8− cells, leading to the development of cytotoxic T lymphocytes (CTL) and T helper cells, and in the apoptotic processes in T cells. See S. Apasov et. al., Immunol. Rev. 146, 5 (1995). ATP serves as a source of extracellular adenosine, as a phosphate donor, and as a transmembrane signaling ligand in both T-cell development and effector functions. It has also been suggested that extracellular ATP and extracellular phosphorylation are involved in cell-to-cell contact leading to lymphocyte activation. The presence of ecto-ATPase and Ag-receptor-induced accumulation of extracellular ATP has been demonstrated for both T helper and CTL. Moreover, apoptosis of thymocytes by ATP is Ca²⁺ independent, suggesting involvement of P_{2X} receptors. S. Apasov et. al., supra.

A P_{2X} receptor identified in muscle and neuronal cells was found to be highly homologous to the RP-2 gene, which is expressed in apoptotic thymocytes. Furthermore, it has been suggested that abnormal signaling through purinoceptors by extracellular adenosine (which is accumulated because of cell surface-associated ADA deficiency) may cause the apoptosis of T cells in ADA SCID.

Human Immunodeficiency Virus (HIV), formerly and occasionally referred to as lymphadenopathy-associated virus (LAV), human-T-lymphotropic virus (HTLV), or acquired immune deficiency syndrome (AIDS)-related virus (ARV), is generally recognized as causing acquired immunodeficiency syndrome, or AIDS. At least two HIV viruses, HIV-1 and HIV-2, have been identified as AIDS infective agents. Levels of ADA isoenzyme levels in sera of patients with AIDS are higher than those in healthy controls, while ADA activity in infected cells is promoted by HIV-1 infection. I. Tsuboi, Clin. Diag. Lab. Immunol. 2, 626, (1995).

HIV is cytopathic for T lymphocytes expressing CD4 (OKT 4) antigen, but not OKT 8. Both adenosine and HIV decrease the expression of CD4 antigen on the cell surface of human T cells. The HIV genome contains a polyadenylated 3′ end that can contact adenosine receptors on human leukocytes. HIV virions may contact the adenosine receptors of cells surface in certain steps of the infection. The adsorption of virus to its cellular receptor (CD4 antigen) can indirectly activate adenosine receptors resulting in a decrease of CD4 expression, which is regarded as an adenosine receptor-related phenomenon. Therefore, pretreatment of cells with adenosine, and the activation of A₂ receptors, reduces the expression of CD4 antigens available for the viruses in their binding to the cells. See S. Sipka et al., Acta Biochim. Biophys., Hung. 23, 75 (1988).

Several chemokine receptors have been shown to act as coreceptors for HIV-1 entry into cells of different lineages. CCR5 is expressed in primary monocytes, macrophages, primary T cells, and granulocyte precursors. Individuals with mutations of CCR5 expression show resistance to HIV-1 infection. Agents that increase cAMP down-regulate CCR5 expression in monocyte-derived macrophages and impair the capacity of M-tropic HIV-1 isolates to infect treated cells. M. Thivierge et al., Blood 92, 40 (1998).

During all stages of HIV infection, tissue macrophages provide a unique viral reservoir. In these cells, HIV persistently replicates in the absence of cytopathicity, escapes immune surveillance, and spreads via cell-to-cell contact. It has been suggested that the persistence of HIV in macrophages may be NF-κB dependent. NF-κB is a heterodimeric protein and transcription factor, anchored in the cytosol by an inhibitory protein, IκBα. Following cell activation by a number of extracellular stimuli, IκBα undergoes a hyperphosphorylation event that renders the molecule susceptible to degradation. This process results in the release of NF-κB, which undergoes nuclear translocation and drives gene transcription. In the absence of exogenous cellular activation, human macrophages express constitutive levels of NF-κB in nuclei. Persistent HIV replication in human macrophages or monocytes upregulates NF-κB activity. The half-life of IκBα in HIV-infected cells is reduced by at least 50% compared to that in uninfected cells, which directly correlates with increased levels of the nuclear pool of NF-κB in HIV-infected cells. The Iκκ complex kinase activity is selectively activated, and is shown to mediate increased NF-κB activation in HIV-infected cells. See S. Asin et al., J. Virology 73, 3893 (1999).

The mechanism whereby HIV infection induces activation of NF-κB in cells of monocyte lineage remains unknown. It has been reported that activation of protein kinase C is an essential component of NF-κB mediated HIV infection. See S. Asin et al., supra. It has also been reported that CD4 glycoprotein, expressed in the surface of T helper cells and macrophages, is required for high affinity binding of HIV viral envelope glycoprotein to target cells and subsequent viral entry. Moreover, it has been shown that entry of primate lentiviruses into target cells is dependent upon the interaction of the viral envelope glycoprotein with CD4 and one or more members of the G protein-coupled receptor (GPCR) family of transmembrane proteins. See Unutmaz et al., Immunology 10, 225 (1998). Understanding the mechanism to inhibit HIV virus-induced activation of NF-κB may decrease viral persistence in these cells and eliminate them as a potential reservoir of HIV replication in infected patients.

Adenosine receptors are members of the superfamily of GPCRs. Four subtypes, referred to as the A₁, A₂ₐ, A_{2b}, and A₃ adenosine receptors, are currently recognized. See Olah and Stiles, Pharmacol. Ther. 85, 55 (2000). A₁ adenosine receptors are coupled via G proteins to a number of effector systems, including adenylate cyclase, phospholipase A₂ (PLA₂), phospholipase C (PLC), potassium channels, calcium channels, and guanylate cyclase. Olah and Stiles, supra; van Galen et al., Medicinal Res. Rev. 12, 423 (1992); Akbar et al., Molecular Pharmacol. 45, 1036 (1994). A₁ adenosine receptors inhibit adenylate cyclase and stimulate PLC and PLA₂ by coupling to a pertussis toxin-sensitive inhibitory G protein (Gᵢ). Furthermore, activation of A₁ adenosine receptors increases protein kinase C activity in coronary arteries. See Marala and Mustafa, Am. J. Physiol. 268, H271 (1995).

In different cell types, lipopolysaccharide (LPS) responses, such as cytokine release, are linked to a number of signal transduction pathways (e.g., PLA₂, PLC and adenylate cyclase), phosphorylation of proteins (e.g., protein kinase C and protein kinase A), and activation of transcription factors (e.g., NFκB). See Chen et al., Current Topics in Microbiol. and Immunol. 181,169 (1992); Schletter et al., Arch. Microbiol. 164, 383 (1995); Sweet and Hume, J. Leukoc. Biol. 60, 8 (1996). The inhibitory effect of LPS on adenylate cyclase or activation of PLA₂ in macrophages is pertussis toxin sensitive, suggesting that the protein responsible for LPS activation of these signal transduction pathways is coupled to a Gᵢ protein. See Chen et al., supra; Jakway and DeFranco, Science 234, 743 (1986); Coffee et al., Biochim. Biophys. Acta. 1035, 201 (1990). By coupling to a Gᵢ protein, LPS inhibits adenylate cyclase and activates the production of arachidonic acid metabolites.

In normal human monocytes and cultured cells, heterotrimeric G proteins specifically regulate CD14-mediated, LPS-induced mitogen-activated protein kinase (MAPK) activation and cytokine production. Solomon, et al., J. Clin. Invest. 102, 2019 (1998). Also, LPS-induced IL-1 production in the human promonocytic cell line U937, is linked to de novo synthesis of an inhibitory G protein. Daniel-Issakan et al., J. Biol. Chem. 264, 20240 (1989). Moreover, the effect of LPS on arachidonic acid metabolism and TXA₂ release in rat peritoneal macrophages was found to be dependent on protein kinase C activation. Geisel et al., Biochemica. et Biophysica. Acta. 1085,15 (1991).

It has been reported that LPS is a potent stimulator of the expression of HIV-1 in monocytes and macrophages. See Pomerantz et al., J. Exp. Med. 172, 253 (1990). It has also been reported that LPS-induced HIV-1 expression in transgenic mice is mediated by TNF-α and IL-1. See Tanaka et al., AIDS 14,1299 (2000). In that particular study, HIV-1 gene expression was activated 10-20 fold by LPS and serum p24 Gag protein levels reached 400 pg/ml, similar to those in the serum of AIDS patients. Moreover, in humans with endotoxemia, (myco)bacterial antigens, including LPS, cell wall components of Mycobacterium tuberculosis (lipoarabinnomannan) or Staphylococcus aureus (lipoteichoic acid), or staphylococcal enterotoxin B increased expression of HIV co-receptors CXCR4 and CCR5 on CD4+ Tcells. See Juffermans et al., Blood 96, 2649 (2000). These effects of LPS on the expression of HIV-1 in monocytes and macrophages, and expression of chemokine HIV co-receptors on T cells may be mediated by A₁ adenosine receptors and P_{2X} purinoceptors. The present inventor has previously discovered that LPS binds to and activates A₁ adenosine receptors on human pulmonary artery endothelial cells (unpublished observations). Periodate oxidized adenosine 5′-triphosphate (o-ATP), a P_{2X} purinoceptor antagonist, was found to inhibit the effects of LPS on nitric oxide (NO) production, inhibit the expression of inducible nitric oxide synthase (iNOS), and inhibit NF-kappa-B-like binding activity in RAW 264.7 macrophages. Hu, et al., J. Biol. Chem. 273, 27170 (1998). Finally, the P_{2X} purinoceptor antagonist, pyridoxal-phosphate-6-azophenyl-2′,4′-disulphonic acid (PPADS) was found to prevent the effect of LPS on the expression of iNOS.

### SUMMARY OF THE INVENTION

It has now been found that administration of compositions comprising A₁ adenosine receptor antagonists and/or P_{2X} purinoceptor antagonists, or a combination thereof, can prevent or inhibit immune system disorders. Although the Applicant does not wish to be bound to any particular theory of the invention, it is believed that A₁ adenosine receptor antagonists prevent or delay the entry of HIV virus into cells. A₁ adenosine receptor antagonists also appear to prevent HIV-induced upregulation of chemokine receptors in monocytes, macrophages and T cells; prevent activation of NF-κB in monocytes and macrophages; prevent activation of nuclear A1 adenosine receptors and nuclear PKC in the spleen; and prevent HIV-1 gene expression in the spleen.

Moreover, ATP may serve as a contact-to-contact mediator for monocytes/macrophages and T cells, and aid in the infection of these cells with HIV by serving as a phosphate donor. ATP may also upregulate chemokine coreceptors for HIV on these cells via P_{2X} purinoceptor activation.

In view of the foregoing, certain aspects of the present invention relate to methods for treating an immune system disorder in a subject in need of such treatment. In other aspects, the present invention relates to methods for preventing an immune system disorder in a subject in need of such treatment. In one embodiment, the method comprises administering to the subject an A₁ adenosine receptor antagonist in an amount effective to treat a disorder of immune deficiency. In another embodiment, the method comprises administering to the subject an A₁ adenosine receptor antagonist in an amount effective to prevent an immune system disorder. In preferred embodiments, the immune system disorder is HIV infection or AIDS. In other preferred embodiments, the immune system disorder is adenosine deaminase deficiency-dependent severe combined immunodeficiency (ADA SCID).

The present inventor has further discovered that administration of a P_{2X} purinoceptor antagonist is useful as a treatment for immune system disorders. The present inventor has also discovered that administration of a P_{2X} purinoceptor antagonist is useful in methods of preventing immune system disorders. Thus, certain embodiments of the invention relate to methods of treating an immune system disorder in a subject in need of such treatment, the method comprising administering to a subject a P_{2X}purinoceptor antagonist in an amount effective to treat the immune system disorder. Other embodiments of the invention relate to methods of preventing an immune system disorder in a subject in need of such treatment, the method comprising administering to a subject a P_{2X}purinoceptor antagonist in an amount effective to prevent the immune system disorder. In preferred embodiments, the immune system disorder is HIV infection or AIDS. In other preferred embodiments, the immune system disorder is adenosine deaminase deficiency-dependent severe combined immunodeficiency (ADA SCID).

The present invention further provides a method of treating certain disorders of the immune system by administering an effective amount of a composition or compound comprising at least one A₁ adenosine receptor antagonist and at least one P_{2X} purinoceptor antagonist. In certain embodiments of the invention, a compound administered to prevent or treat an immune disorder is both an A₁ adenosine receptor antagonist and a P_{2X} purinoceptor antagonist.

As an additional aspect, the present invention provides pharmaceutical formulations for the treatment of immune disorders comprising an A₁ adenosine receptor antagonist, and/or a P_{2X} purinoceptor antagonist, or a combination thereof, together with a pharmaceutically acceptable carrier.

The foregoing and other aspects of the present invention are explained in detail in the description set forth below.

### DETAILED DESCRIPTION OF THE INVENTION

The present invention will now be described with reference to the accompanying figures and specification, in which preferred embodiments of the invention are illustrated. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only, and is not intended to be limiting of the invention. As used in the description of the invention and the appended claims, the singular forms “a”, “an” and “the” are intended to include the plural forms as well, unless the context clearly indicates otherwise. All publications, patent applications, patents, and other references mentioned herein are incorporated by reference in their entirety.

The methods and formulations of the present invention are useful in treating disorders of the immune system (i.e., immunodeficiencies). Immunodeficiencies are generally categorized as either acquired immunodeficiencies or inherited immunodeficiencies. Acquired immunodeficiencies include human immunodeficiency virus-1 (HIV-1) infection, herpes virus infections, Epstein-Barr virus infections, lepromatous leprosy and diminished immune capacity resulting from skin burns in burn patients (i.e. burn-related immunodeficiency). Inherited immunodeficiencies include several genetically different forms of SCID, including adenosine deaminase deficiency dependent SCID (ADA SCID), SCID autosomal recessive with and without B cells (no ADA deficiency), SCID X-linked recessive without B cells, SCID autosomal recessive (with ADA deficiency), purine nucleotide phosphorylase deficiency (PNP SCID), severe combined immune deficiency (IL-2 receptor deficiency) (i.e. X-linked SCID), and bare lymphocyte syndrome. Other immunodeficiencies include various forms of congenital or genetically determined hematopoietic abnormalities, several high-risk leukemias and several forms of severe life-threatening aplastic anemia. Still other immunodeficiencies that may be treated by methods and formulations of the present invention include Wiskott-Aldrich syndrome; Blackfan-Diamond syndrome; Fanconi anemia; severe neutrophil dysfunction; chronic granulomatous disease of childhood; severe (Kostman-type) agranulocytosis; immunodeficiency and neutropenia of cartilage-hair hypoplasia; infantile and late onset osteopetrosis; aplastic anemia (toxic chemical, idiopathic, immunological, and non-Fanconi genetic); acute myeloid leukemia; chronic myeloid leukemia; Burkitt lymphoma, and recurrent acute lymphatic leukemia.

In preferred embodiments of the invention, the immune system disorder that is treated or prevented is HIV infection or AIDS. In other preferred embodiments, the immune system disorder that is treated or prevented is adenosine deaminase deficiency-dependent severe combined immunodeficiency (ADA SCID).

A₁ adenosine receptor antagonists and P_{2X} purinoceptor antagonists are referred to herein as “active compounds.” As used herein, “purinoceptor,” “purinergic receptor,” and “purinoreceptor” are used interchangeably.

Numerous A₁ adenosine receptor antagonists are known to those of skill in the art. One known class of adenosine receptor antagonist is the xanthine family, which include caffeine and theophylline. See e.g., Müller et al., J. Med. Chem. 33, 2822 (1990). Numerous A₁ adenosine receptors antagonists have been synthesized. For example, 1,3-dipropyl-8-cyclopentylxanthine (DPCPX) is a highly selective A₁ adenosine receptor antagonist with negligible nonspecific binding (less than 1%) in tissues. See Jacobson et al., J. Med. Chem. 35, 407 (1992); Bruns, RF “Adenosine Receptor Binding Assays”, in Receptor Biochemistry and Methodology, Volume II: Adenosine Receptors, DMF Cooper and C. Londos (Eds.), Alan Liss, Inc., New York, N.Y. (1988), pp. 43-62. Other examples of A₁ adenosine receptor antagonists include, but are not limited to, xanthine amine congener (XAC); xanthine carboxylic congener (XCC); 1,3-dipropyl-xanthines such as 1,3-dipropyl-8-(−3-noradamantyl) xanthine (KW 3902), 1,3-dipropyl-8-(dicyclopropylmethyl) xanthine (KF 15372), 1,3-dipropyl-8-[2-(5,6-epoxy)norbonyl]xanthine (ENX), 8-(1-aminocyclopentyl)-1,3-dipropylxanthine (IRFI 117), 1,3-dipropyl-8-(3-noradamantyl) xanthine (NAX) and 1,3-dipropyl-8-(3-oxocyclopentyl) xanthine (KFM 19); 1-propyl-3-(4-amino)-3-phenethyl)-8-cyclopentylxanthine (BW-A844U); 1,3-dipropyl-8-sulfophenylxanthine (DPSPX); cyclopentyl theophylline (CPT) and 7-[2-ethyl (2-hydroxyethyl)amino]-ethyl]-3,7-dihydro-1,3-dimethyl-8-(phenylmethyl)-1H-purine-2, 6-dione (Bamifylline); N⁶, 9-methyl adenines such as (+)-N⁶-endonorbornan-2-yl-9-methyladenine (N-0861) and 8-(N-methylisopropyl) amino- N⁶-(5′-endohydroxy-endonorbornyl)-9-methyladenine (WRC-0571); N⁶, 9-disubstituted adenines; 2-phenyl-7-deazaadenines such as (R)-7,8-dimethyl-2-phenyl-9-(1-phenylethyl)-7-deazaadenine; 7,8-dihydro-8-ethyl-2-(3-noradamantyl)-4-propyl-1H-imidazo[2,1-i]purin-5(4H)-one; (+)R-1-[(ε)-3[2-[phenylpyrazolo (1,5-a) pyridin-3-yl]acryloyl]-2-piperidine ethanol; 8-azaxanthines such as 7-cyclopentyl-1,3-dipropyl-8-azaxanthine; tetrahydrobenzothiophenones such as ethyl-3-(benzylthio)-4-oxo-4,5,6,7-tetrahydrobenzo[c]thiophene-1-carboxylate; N-6-cyclopentyl-3′-substituted xylo-furanosyl adenosines. See Van Calinbergh, J. Med. Chem. 40, 3765 (1997).

Additional A₁ adenosine receptor (A₁AR) antagonists are set forth in U.S. patent application Ser. No. 08/753,048, filed Nov. 19, 1996 (now U.S. Pat. No. 5,786,360 to Neely, issued Jul. 28, 1998), incorporated herein by reference in its entirety. Additional A₁AR antagonists useful in the practice of the present invention include those set forth in, for example, U.S. Pat. Nos. 5,599,671 to Jacobson et al.; 5,998,387 to Belardinelli; 5,066,655, to Olsson; 5,298,508 to Jacobson et al.; 4,696,932 to Jacobson et al.; 5,773,530 to Akahane et al.; 5,565,566, to Olsson; 5,668,139 to Belardinelli et al.; 5,446,046 to Bellardinelli et al.; and 5,310,916 to Jacobson et al. (all of which are incorporated herein by reference).

Still other A₁AR antagonists useful in the practice of the present invention include those set forth in, for example, published PCT Applications WO 97/24363A1 to Belardinelli et al.; WO 95/11904A1 to Belardinelli et al.; and WO 88/08303A1 to Olsson; European Patent Application No. EP 764647 to Connell et al.; Australian Patent Application Nos. AU1044995A1, AU699630B2, and AU728439B2, all to Belardinelli et al.; Japanese applications JP8099976A to Shiokawa et al. and JP9216883A to Kuroda; and Chinese Application No. CN1206420A to Belardinelli et al. (all of which are incorporated herein by reference).

Additionally, selective analogs of adenosine receptor antagonists have been developed through the “functionalized congener” approach. Analogs of adenosine receptor ligands bearing functionalized chains have been synthesized and attached covalently to various organic moieties such as amines and peptides. Jacobson et al., J. Med. Chem. 35, 408 (1992) has proposed various derivatives of adenosine and theophylline for use as receptor antagonists.

Antibodies raised against the A₁ adenosine receptor that selectively target and bind to the receptor can also be used as A₁ adenosine receptor antagonists. Such antibodies targeted to the A₁ adenosine receptor can be produced routinely in accordance with well known methods of antibody production. As used herein, the term “A₁ adenosine receptor antagonist” encompasses antibodies that selectively or specifically bind to the receptor, when such antibodies are used for their antagonist effects. The term “antibody,” as used herein, includes polyclonal antibodies, monoclonal antibodies, and active fragments or polypeptides thereof. Antibodies to the A₁AR are set forth in, for example El-Etr, Neuro. Sci. Lett. 145, 15 (1992); Ku et al., J. Immunol. 139, 2376 (1987); Salmon et al., J. Immunol. 151, 2775 (1993); and U.S. Pat. No. 5,144,010 to Erlanger and Cleveland.

P_{2X} purinoceptor antagonists are also known in the art. An example of a selective P_{2X} purinoceptor antagonist is pyridoxalphosphate-6-azophenyl-2′,4′-disulfonic acid (PPADS). Additional specific pharmacological antagonists of purinoceptors have been described by Humphrey et al., Naunyn-Schmied. Arch. Pharmacol. 352, 585 (1995); Abracchio and Burnstock, Pharmacol. Ther. 64, 445 (1994); Bultmann et al., Naunyn-Schmied. Arch. Pharmacol. 354, 481 (1996); and Bultmann et al., Naunyn-Schmied. Arch. Pharmacol. 354, 498 (1996). Antibodies raised against the P_{2X} purinoceptor that selectively target and bind to this receptor can also be used as P_{2X} purinoceptor antagonists. Such antibodies targeted to the P_{2X} purinoceptor can be produced routinely in accordance with well known methods of antibody production. As used herein, the term “P_{2X} purinoceptor antagonist” encompasses antibodies that selectively or specifically bind to the receptor, when such antibodies are used for their antagonist effects. As set forth above, the term “antibody,” as used herein, includes polyclonal antibodies, monoclonal antibodies, and active fragments or polypeptides thereof.

An active compound of the present invention may optionally be provided and administered in the form of a free base, or may be in the form of a pharmaceutically acceptable salt thereof. Suitable pharmaceutically acceptable salts include inorganic acid addition salts such as hydrochloride, hydrobromide, sulfate, phosphate, and nitrate; organic acid addition salts such as acetate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt and N,N-dibenzylethylenediamine salt; and salts with basic amino acid such as lysine salt and arginine salt.

The present invention provides methods of preventing and treating disorders of the immune system, wherein an effective amount of an A₁ adenosine receptor antagonist, and/or a P_{2X} purinoceptor antagonist, or a combination thereof, is administered to a subject in need of such treatment. A single compound that antagonizes both the A₁ adenosine receptor and the P_{2X}purinoceptor may also be used in the methods of the present invention.

By the terms “treating”or “treatment” of an immune system disorder, it is intended that the severity of the disorder or the symptoms of the disorder are reduced, or the disorder is partially or entirely eliminated, as compared to that which would occur in the absence of treatment. Treatment does not require the achievement of a complete cure of the disorder.

By the terms “preventing” or “prevention” of the immune system disorder, it is intended that the inventive methods eliminate or reduce the incidence or onset of the disorder, as compared to that which would occur in the absence of treatment. Alternatively stated, the present methods slow, delay, control, or decrease the likelihood or probability of the disorder in the subject, as compared to that which would occur in the absence of treatment.

An “effective amount” is that amount able to reduce the severity, development, or onset of the disorder that would occur in the absence of the antagonists, or slow the progress (over time) of the disorder, compared to that which would occur in the absence of said antagonists. The term “effective amount” also refers to a concentration of an A₁ adenosine receptor antagonist, P_{2X} purinoceptor antagonist, or combination thereof, which is sufficient to interfere with pathological changes caused by the disorder. Preferably, the A₁ adenosine receptor antagonist is a selective A₁ adenosine receptor antagonist. Also preferably, the P_{2X} purinoceptor antagonist is a selective P_{2X} purinoceptor antagonist.

The therapeutically effective dosage of any specific compound, the use of which is in the scope of the present invention, will vary somewhat from compound to compound, patient to patient, and will depend upon the condition of the patient and the route of delivery. As a general proposition, a dosage from about 0.1 to about 20 mg/kg body weight will have therapeutic efficacy, with still higher dosages potentially being employed for oral and/or aerosol administration. Toxicity concerns at the higher level may restrict intravenous dosages to a lower level such as up to about 10 mg/kg, all weights being calculated based upon the weight of the active base, including the cases where salt is employed. Typically a dosage from about 0.56 mg/kg to about 5 mg/kg will be employed. In certain circumstances, higher or lower doses may be also appropriate. The daily dose can be administered either by a single dose in the form of an individual dosage unit or several smaller dosage units, by multiple administration of subdivided dosages at certain intervals, or by a continuous infusion.

The methods of the present invention may be carried out in conjunction with other therapies for the immune system disorder that is being treated or prevented. For example, pharmaceutical compositions known to be useful in the treatment of HIV infection and AIDS may be administered concurrently with the A₁ antagonists or P_{2X} purinoreceptor antagonists of the present invention. Alternatively, a course of treatment known to be useful in the treatment of HIV infection and AIDS may be carried out while a course of treatment utilizing the present invention is also carried out.

The present invention also provides pharmaceutical formulations, both for veterinary and for human medical use, which comprise the active compounds of the invention, together with one or more pharmaceutically acceptable carriers thereof and optionally any other therapeutic ingredients. The carrier(s) must be pharmaceutically acceptable in the sense of being compatible with the other ingredients of the formulation and not unduly deleterious to the recipient thereof. Pharmaceutically acceptable carriers include, but are not limited to, saline, water, dextrose and water, cyclodextrins or similar sugar solutions, low dose sodium hydroxide solutions, propylene glycol, and polyethylene glycol.

The formulations of the present invention may be suitable for inhalation (e.g., as an aerosol), oral, rectal, topical, nasal, ophthalmic, parenteral (including but not limited to subcutaneous, intramuscular, intravenous, and intraarterial), intraarticular, intrapleural, intraperitoneal, vaginal, bladder instillation, and intracerebral (alternatively, into the cerebral spinal space) administration. Formulations suitable for oral, inhalation, and parenteral administration are preferred.

The formulations may conveniently be presented in unit dosage form and may be prepared by any of the methods well known in the art of pharmacy. All methods include the step of bringing the active compound into association with a carrier which constitutes one or more accessory ingredients. In general, the formulations are prepared by uniformly and intimately bringing the active compound into association with a liquid carrier, a finely divided solid carrier, or both, and then, if necessary, shaping the product into desired formulations.

Formulations of the present invention suitable for oral administration may be presented as discrete units such as capsules, cachets, tablets or lozenges, each containing a predetermined amount of the active compound as a powder or as granules; or a non-aqueous liquid such as a syrup, an elixir, an emulsion or a draught.

A tablet may be made by compression or molding, optionally with one or more accessory ingredients. Compressed tablets may be prepared by compressing in a suitable machine, with the active compound being in a free-flowing form such as a powder or granules which is optionally mixed with a binder, disintegrant, lubricant, inert diluent, surface active agent or dispersing agent. Molded tables comprised of a mixture of the powdered active compound with a suitable carrier may be made by molding in a suitable machine.

Formulations suitable for parenteral administration conveniently comprise a sterile aqueous preparation of the active compound, which is preferably isotonic with the blood of the recipient and pyrogen-free.

In addition to the aforementioned ingredients, the formulations of this invention may further include one or more accessory ingredient(s) selected from diluents, buffers, flavoring agents, binders, disintegrants, surface active agents, thickeners, lubricants, preservatives (including antioxidants) and the like.

In yet another aspect of the present invention, there is provided an injectable, stable, sterile composition comprising an active compound or compounds of the present invention, in a unit dosage form in a sealed container. The compound or salt is provided in the form of a lyophilizate which is capable of being reconstituted with a suitable pharmaceutically acceptable carrier to form a liquid composition suitable for injection thereof into the subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidylcholine.

Further, the present invention provides liposomal formulations of the compounds of present invention. The technology for forming liposomal suspensions is well known in the art. When the compound is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques.

The following Examples are provided to illustrate the present invention, and should not be construed as limiting thereof. Examples 1 and 2 are carried out generally according to the methods set forth in Jackson et al., J. Clin. Microbiol. 26,1416 (1988). Examples 3, 4, and 5 are carried out generally as set forth in Heredia, et al., J Acquired Immune Deficiency Syndromes 25, 246 (2000); St. Clair, et al., Science 253,1557 (1991); and Asin, et al., J. Virol. 73, 3893 (1999)

### EXAMPLE 1

### Viral Culture Method for HIV-1: Isolation of PBMC From Normal (HIV-1 Negative) Donors

PBMC are obtained from the buffy coats of whole-blood donors negative for HIV-1 antibodies. Each buffy coat is diluted 1:3 with sterile phosphate-buffered saline (pH 7.3 at 24° C.) within eight hours of donation. Thirty milliliters of diluted buffy coat is layered over 15 ml of sterile Ficoll-Paque (Pharmacia, Inc., Piscataway, N.J.) and centrifuged at 350×g for 30 to 45 minutes at room temperature. The layer containing the PBMC is removed and washed twice in sterile phosphate-buffered saline. Pelleted cells are suspended and pooled in stimulation medium (fresh RPMI 1640 medium) [GIBCO Laboratories, Grand Island, N.Y.,] containing 20% heat-inactivated fetal bovine serum (FBS) [GIBCO], two mM glutamine, four μg of Polybrene [Sigma Chemical Co., St. Louis, Mo.] per ml, 200 U of penicillin per ml, 200 μg of streptomycin per ml, and four μg of phytohemagglutinin-P [Sigma] per ml) and placed in upright 275-ml tissue culture flasks at a concentration of 10⁶ cells per ml. After two to four days in culture at 37° C. in a 5% CO₂ atmosphere, the supernatant above the settled cells is removed to bring the volume to one-fourth that of the original. Unwashed samples of 5×10⁶ or 3×10⁶ of these donor PBMCs are used to feed cultures of PBMCs from patients HIV-1 antibody positive.

### EXAMPLE 2

### Viral Culture Method for HIV-1: Isolation of PBMC From Blood of HIV-Antibody Positive Patients

PBMC are obtained from whole-blood of patients positive for HIV-1 antibodies. For separation of PBMC from patients, 20 to 30 ml of heparinized blood is diluted 1:3 with sterile phosphate-buffered saline within 24 h collection. Thirty-milliliter portions of diluted blood are layered over 15 ml of sterile Ficoll-Paque and centrifuged at 350×g for 30 to 45 min at room temperature. The layer containing the PBMC is removed and washed twice in sterile phosphate-buffered saline. Pelleted cells are suspended and pooled in 10 ml of T-cell growth factor medium (fresh RPMI 1640 medium containing 20% heat-inactivated FBS, 5% interleukin-2 (IL-2) [Cellular Products, Buffalo, N.Y.], 2 mM glutamine, 5 μg of Polybrene per ml, 200 U of penicillin per ml, and 200 μg of streptomycin per ml).

PBMCs from normal (HIV-1 negative) subjects are stimulated with 2.5 mg/ml phytohemagglutinin-P (PHA) (Boehringer Mannheim, Indianapolis, Ind.) for three days. A cell suspension volume equal to 10⁷ PBMC from HIV-antibody positive patients is placed in an upright 50-ml flask along with 5×10⁶ PHA-stimulated donor PBMC. The final cell suspension volume is diluted to 15 ml with T-cell growth factor medium. These co-cultures are incubated at 37° C. in a 5% CO₂ atmosphere for as long as 28 days. Approximately seven ml of culture medium above the settled cells is removed every three to four days for HIV-1 p24 antigen detection and replaced with an equal volume of fresh T-cell growth factor medium. An additional 3×10⁶ PHA-stimulated donor PBMC are added every seven days.

### EXAMPLE 3

### Effect of A₁ Adenosin Receptor Antagonist, P_{2X} Purinoceptor Antagonist, and A₁ Adenosine Receptor Antagonist Plus a P_{2X} Purinoceptor Antagonist on HIV-1 Replication

PHA-stimulated donor PMBCs (10⁷) are sedimented by low speed centrifugation and resuspended in 10 ml infective cell-free supernatant from cell cultures of PBMCs from HIV-antibody positive patients co-cultured with PBMCs from normal subjects containing 360 ng of p24/ml for two hours. Mock-infected cells are used as controls. PBMCs are then washed three times with PBS and cultured in 5% CO₂ at 37° C., in RPMI-1640/10% FBS supplemented with 10 U/ml IL-2. PBMCs are seeded in 96-well flat-bottom plates at a density of 2×10⁵ PBMCs/200 μl. Mock-infected cells are treated with IL-2 alone. Infected PBMCs are treated with (1) IL-2 alone (controls), (2) an A₁ adenosine receptor antagonist (L-97-1, 1 nM-10 μM), (3) a P_{2X} purinoceptor antagonist, such as pyridoxalphosphate-6-azophenyl-2′,4′ disulfonic acid (PPADS) (5-50 μM), or (4) L-97-1 (1 nM-10 μM) plus PPADS (5-50 μM). Following three days of culture, one half of the medium is replaced with fresh medium containing IL-2 alone (mock-infected and controls) or IL-2 plus treatments for infected cells. After ten days of culture, HIV-1 p24 antigen production in the cell culture supernatant is assayed. Experiments for mock-infected cells and infected cells for controls (IL-2 alone) and for each treatment (L-97-1, PPADS, or L-97-1 plus PPADS) are performed with three different batches of cells and two serial supernatants are assayed for HIV p24 antigen in duplicate.

### EXAMPLE 4

### Detection of HIV Antigen

Culture supernatant fluids are tested for the presence of p24 core antigen of HIV with the use of an enzyme-linked immunosorbent assay (Abbott Laboratories, North Chicago, ll.) Culture supernatants are removed every three to four days and frozen in a one-mL aliquots at −20° C. Specimens are thawed within one month of collection and tested according to the manufacturers directions.

### EXAMPLE 5

### Analysis of Data

Statistical analysis of the data is performed with the use of the Student's t test for unpaired data for each concentration of L-97-1, PPADS, or L-97-1 plus PPADS (treatment) versus control (IL-2 alone). P<0.05 is accepted as statistically significant.

The foregoing is illustrative of the present invention and is not to be construed as limiting thereof. The invention is defined by the following claims, with equivalents of the claims to be included therein.

That which is claimed:

1. A method for treating an immune system disorder in a subject in need of such treatment, comprising administering to said subject a compound selected from the group consisting of:
(a) A₁ adenosine receptor antagonists;
(b) P_{2X} purinoceptor antagonists; and
(c) a combination of at least one A₁ adenosine receptor antagonist and at least one P_{2X} purinoceptor antagonist;
wherein said compound is administered in an amount effective to treat the immune system disorder.

2. The method of claim 1 wherein the disorder is selected from the group consisting of HIV infection, AIDS, and adenosine deaminase deficiency-dependent severe immunodeficiency disease (ADA SCID).

3. A method according to claim 1, wherein the A₁ adenosine receptor antagonist is an antibody that binds the A₁ adenosine receptor.

4. A method according to claim 1, wherein the P_{2X} purinoceptor antagonist is an antibody that binds the P_{2X} purinoceptor.

5. A method for preventing or delaying the onset of an immune system disorder in a subject in need of such treatment, comprising administering to said subject a compound selected from the group consisting of:
(a) A₁ adenosine receptor antagonists;
(b) P_{2X} purinoceptor antagonists; and
(c) a combination of at least one A₁ adenosine receptor antagonist and at least one P_{2X} purinoceptor antagonist;
wherein said compound is administered in an amount effective to prevent or delay the onset of the immune system disorder that would occur in the absence of the administration.

6. The method of claim 5 wherein the disorder is selected from the group consisting of HIV infection, AIDS, and adenosine deaminase deficiency-dependent severe immunodeficiency disease (ADA SCID).

7. The method according to claim 5, wherein the A₁ adenosine receptor antagonist is an antibody that binds to the A₁ adenosine receptor.

8. A method according to claim 5, wherein the P_{2X} purinoceptor antagonist is an antibody that binds to the P_{2X} purinoceptor.

9. A method for treating HIV infection or AIDS in a subject in need of such treatment, comprising administering to said subject a compound selected from the group consisting of:
(a) A₁ adenosine receptor antagonists;
(b) P_{2X} purinoceptor antagonists; and
(c) a combination of at least one A₁ adenosine receptor antagonist and at least one P_{2X} purinoceptor antagonist;
wherein said compound is administered in an amount effective to treat the HIV infection or AIDS.

10. The method of claim 9, wherein the treatment is carried out in conjunction with another treatment for HIV infection or AIDS.

11. A method according to claim 9, wherein the A₁ adenosine receptor antagonist is an antibody that binds to the A₁ adenosine receptor.

12. A method according to claim 9, wherein the P_{2X} purinoceptor antagonist is an antibody that binds to the P_{2X} purinoceptor.

13. A method for preventing or delaying the onset of HIV infection or AIDS in a subject in need of such treatment, comprising administering to said subject a compound selected from the group consisting of:
(a) A₁ adenosine receptor antagonists;
(b) P_{2X} purinoceptor antagonists; and
(c) a combination of at least one A₁ adenosine receptor antagonist and at least one P_{2X} purinoceptor antagonist;
wherein said compound is administered in an amount effective to prevent or delay the onset of the HIV infection or AIDS that would occur in the absence of the administration.

14. A method according to claim 13, wherein the A₁ adenosine receptor antagonist is an antibody that binds to the A₁ adenosine receptor.

15. A method according to claim 13, wherein the P_{2X} purinoceptor antagonist is an antibody that binds to the P_{2X} purinoceptor.

16. A method for treating adenosine deaminase deficiency-dependent severe immunodeficiency disease (ADA SCID) in a subject in need of such treatment, comprising administering to said subject a compound selected from the group consisting of:
(a) A₁ adenosine receptor antagonists;
(b) P_{2X} purinoceptor antagonists; and
(c) a combination of at least one A₁ adenosine receptor antagonist and at least one P_{2X} purinoceptor antagonist;
wherein said compound is administered in an amount effective to treat adenosine deaminase deficiency-dependent severe immunodeficiency disease (ADA SCID).

17. The method of claim 16, wherein the treatment is carried out in conjunction with another treatment for adenosine deaminase deficiency-dependent severe immunodeficiency disease (ADA SCID).

18. A method according to claim 16, wherein the A₁ adenosine receptor antagonist is an antibody that binds to the A₁ adenosine receptor.

19. A method according to claim 16, wherein the P_{2X} purinoceptor antagonist is an antibody that binds to the P_{2X} purinoceptor.

20. A method for preventing or delaying the onset of adenosine deaminase deficiency-dependent severe immunodeficiency disease (ADA SCID) in a subject in need of such treatment, comprising administering to said subject a compound selected from the group consisting of:
(a) A₁ adenosine receptor antagonists;
(b) P_{2X} purinoceptor antagonists; and
(c) a combination of at least one A₁ adenosine receptor antagonist and at least one P_{2X} purinoceptor antagonist;
wherein said compound is administered in an amount effective to prevent or delay the onset of the adenosine deaminase deficiency-dependent severe immunodeficiency disease (ADA SCID) that would occur in the absence of the administration.

21. A method according to claim 20, wherein the A₁ adenosine receptor antagonist is an antibody that binds to the A₁ adenosine receptor.

22. A method according to claim 20, wherein the P_{2X} purinoceptor antagonist is an antibody that binds to the P_{2X} purinoceptor.
